(19) United States
(12) Patent Application Publication
(10) Pub. No.: US 2024/0225679 A9
(43) Pub. Date: Jul. 11, 2024

(54) ENDOSCOPIC SYSTEM AND METHOD

(71) Applicant: KARL STORZ SE & Co. KG, Tuttlingen (DE)

(72) Inventors: Sebastian WAGNER, Tuttlingen (DE); Sebastian Wenzler, Tuttlingen (DE)

(21) Appl. No.: 18/381,892

(22) Filed: Oct. 19, 2023

(30) Foreign Application Priority Data
Oct. 25, 2022 (DE) 10 2022 128 268.6

(51) Int. Cl. A61B 17/29; A61B 17/00

(52) U.S. Cl. CPC A61B 17/29; A61B 2017/00022; A61B 2017/00398

(57) ABSTRACT

An endoscopic system (10) comprises an actuator (12) at a distal end (14) of the system (10), a manually operable actuating unit (18) at a proximal end (16) of the system (10), for actuating the actuator (12) and choosing an actuator force (F) by means of which the actuator (12) is intended to act on an object (20), the actuating unit (18) being displaceable between a start position and an end position, an actuator motor (24) designed to drive the actuator (12), and a control unit (30) designed to drive the actuator motor (24) initially on the basis of a position sensor (38) without exceeding a limit value (FTH) and then on the basis of a force sensor (40). Further, a method for driving an actuator (12) of an endoscopic system (10) is disclosed.

The present application relates to an endoscopic system and to a method for driving an actuator of an endoscopic system.

A surgeon is primarily reliant on the use of their hands and fingers, which they use to sense, feel, palpate, etc., the tissue during open surgical procedures, and performs many work steps and makes many decisions purely on the basis of their sense of touch.

This sense of touch has been lost more or less in its entirety in conventional manual endoscopic procedures within the scope of minimally invasive surgery (MIS). Although the surgeon feels a resistance, the possibility of making a fine distinction between different tissue types and structures, which they could make intuitively using their hands, is lost, if not fully then at least to a large extent. However, the surgeon is still able to draw conclusions, at least based on optical perception of an image on an electronic visual display. This includes, in particular, a gripping effect that is identifiable in the image, with the result that the surgeon is able to vary the gripping force used to grip the tissue.

This deficiency is amplified in the case of instruments moved purely in motor-driven fashion, since distal instrument movements are implemented purely under path and position control by way of the manual movements at the input. This means that it is not possible anymore to feel any tissue resistance, and the gripping force can at best be monitored only indirectly via the visually observed tissue deformation during the gripping action. However, this becomes very hard or even impossible in practice if the required minimal actuator movements, in particular the jaw part movements, are not controllable by way of a manual input.

The lack of the direct sense of touch is a not insignificant disadvantage of endoscopic procedures, which is why attempts have been made to at least indirectly restore the surgeon's missing sense of touch to the best possible extent, and there have been developments in this respect, ever since endoscopy started to be used. For example, this is implemented by way of mechanical solutions by means of particularly stiff instrument shafts and transfer elements, or by means of mechanisms designed and optimized so that the operating force or finger force at the handle has a 1:1 correspondence with the actuator force acting on the actuator, in particular corresponds to a gripping force acting centrally in the jaw part.

However, it was found in practice that tactile work that feels “real” is not possible, or only possible with difficulties, using the conventional purely mechanical MIS instruments. The user is able to vary and dispense the forces to be exerted by metering their manual exertion of force and can also in principle have at least an approximate idea of resistances from the tissue.

However, sensitively feeling and distinguishing between different tissue types, tissue behaviors, or tissue structures is only possible within limits. Purely on account of varying mechanical transmissions, frictional losses, slip-stick effects, etc., a surgeon requires much practice to be able to work virtually in tactile fashion using these MIS instruments by way of hand-eye control that was trained and learnt over many years.

Relatively recent developments have considered what is known as real force sensory type feedback, the so-called force feedback. In this case, the counterforce exerted by the tissue on the instrument should be measured and this information should be transmitted to the surgeon or user.

To date, there have already been descriptions of various concepts regarding how the counterforce of the tissue can be directly measured (distally), for example by way of force sensors in the actuator, especially in the jaw part, force sensors in the transfer elements, in particular in a tension/push rod, or force sensors on the drive elements such as the actuator motor. However, there also is the option of deriving the actuator force from the drive current for the actuator motor.

Likewise, there are approaches regarding how this information about this counterforce can be conveyed to the user (proximally), for example by creating a counterforce/resistance force in the input, actively by way of drives, for example in motorized, hydraulic or electromagnetic fashion, passively by way of damping or braking mechanisms, or by way of other optical, haptic and/or acoustic information channels, for example imaging, sounder, vibration, etc.

The idea is that the user can use this additional information (counterforce) regarding finely metered gripping movement to sense the tissue, thereby regaining, as it were, their manual tactile capabilities.

Implementing true 1:1 direct feedback is very challenging from a technical point of view, both on the (distal) instrument side with respect to the technical implementation of an exact, precise, reproducible, calibrated force measurement (which is miniaturized on account of limited installation space) and (proximally) on the operating unit with respect to the technical implementation or generation of the measured distal counterforces and, at least currently, still leads to disproportionately complicated and complex technical solutions which provide the user with a sensory force feedback and sense of touch only in rudimentary fashion, if at all.

According to the current prior art/level of knowledge, the actuating unit for controlling the instrument movements, in particular, does not yet really have technical solutions on the horizon which serve to impart a realistic “finger-like sense of touch” that would correspond to the actual gripping/sensing with the fingers.

It is therefore an object of the present invention to provide an improved endoscopic system and an improved method for driving an actuator of an endoscopic system which allow the user to work intuitively with their hand and their fingers despite the known limitations with regard to obtaining a realistic finger-like sense of touch. In the process, the technical implementation in particular should have only comparatively little complexity.

The object is achieved by an endoscopic system comprising an actuator at a distal end of the system, a manually operable actuating unit at a proximal end of the system, for actuating the actuator and choosing an actuator force by means of which the actuator is intended to act on an object, the actuating unit being displaceable between a start position and an end position, an actuator motor designed to drive the actuator to reach a desired position, and a control unit designed to a) actuate the actuator motor depending on a current position of the actuating unit when the actuating unit is actuated between the start position and the end position and if an actuator force exerted by the actuator does not exceed a limit value, b) not actuate the actuator motor any further when the actuating unit is actuated between the start position and the end position and if an actuator force exerted by the actuator reaches or exceeds the limit value, and c) drive the actuator motor beyond the limit value, depending on an operating force exerted on the actuating unit, when the actuating unit is actuated in the end position.

When the actuating unit is actuated from the start position or zero position in the direction of the end position, the actuator is actuated in accordance with the current position of the actuating unit. The operating force exerted by the user on the actuating unit is not decisive here. In particular, a counterforce felt by the user in their hand is only small, or might not even be felt at all. In this case, the actuation of the actuator depends on the current position and not on an operating force exerted by the user. In particular, the actuating unit is actuated between the start position and the end position with a minimal operating force.

However, this actuation is not continued should an actuator force exerted by the actuator reach or exceed the limit value. That is to say, if the limit value is reached or exceeded, then a continued displacement of the actuating unit does not lead to a further actuation of the actuator. Instead, the actuator maintains the current position, and hence usually also the actuator force that it exerts. This type of control is implemented provided the end position has not yet been reached.

The type of control changes once the end position has been reached. A continued or increased exertion of force at the actuating unit now no longer leads to a further displacement of the actuating unit along the path between start position and end position. Instead, the operating force with which the actuating unit is actuated in the end position is sensed. Depending on the sensed operating force, the actuator is actuated further, with the result that the actuator force exerted thereby exceeds the limit value.

In this case, a preferred implementation is set forth below. A new maximum value which exceeds the aforementioned limit value is determined depending on the operating force. Then, the actuator is driven up to this new maximum value. The maximum value is adapted again with a further change in the operating force, and the actuator is once again driven to this new maximum value. The specified limit value, also referred to as initial limit value, is preferably chosen as the value at which the weakest tissue is elastically deformed only to a minimal extent, and no more. The maximum value derived from the operating force then exceeds this limit value.

In preferred configurations, the actuator has a servomotor-type control. The manually operable actuating unit can preferably be situated at a proximal end of the system if the system is designed as a hand-guided instrument. However, the actuating unit can also be positioned as desired in a manner detached from the actual instrument, for example on a control console.

Within the scope of this description, the term proximal should be understood as meaning relating to a side or an end of the system close to the user, where the user controls the operation. If the endoscopic system is embodied as an endoscopic instrument, the proximal end of the system can be in direct mechanical contact with the distal end of the system in particular. Here, this may relate in particular to a hand-guided instrument in which the distal end is also positioned directly by way of the manual guidance at the proximal end. However, the endoscopic system may also be embodied as a robotic system or a remote manipulator.

Within the scope of the invention, the inventors have recognized that, after some practice with surgical procedures using the manual standard systems, a proficient surgeon is able to compensate for the lack of sensory feedback to such an extent that these procedures can be implemented successfully and that ever more endoscopic procedures have become established and gained acceptance in addition to open surgery procedures.

As one aspect of the solution, the inventors have identified that although the surgeon does not have sensory feedback using these manual standard systems, they are able to vary the actuator force that is applied to the tissue and are able to meter said force on the basis of the visually observed effect on the tissue. That is to say although the surgeon is hardly able to feel by way of the system mechanism, they are able to control the forces via the operating force or manual force and adapt said forces accordingly by way of the visual observation of the effect.

This functionality of force control is no longer given in the case of a servomotor-based path conversion of the finger movement to the actuator, in particular to jaw part movements, which makes the handling of the systems with servomotor-based path conversion significantly more difficult in comparison with manually actuated standard MIS systems.

Hence, the invention in particular offers the surgeon performing an endoscopic operation the option of having the same functionality for system movements driven by a servomotor as is available for manually actuated systems. In so doing, the surgeon should especially be provided with the option of varying the effective actuator force and of adapting the forces in real time on the basis of the visually observed effect.

The technical solution to this end should enable only force variation and force metering control in a targeted fashion, and quite deliberately exclude the object of a tactile force sensory type feedback. This simplifies the technical implementation but surprisingly nevertheless leads to an advantageous operability.

This enables a simple and robust technical solution which quite deliberately avoids the complex complicated technical solutions for sensory feedback that is as real as possible. The inventors have recognized that these currently known solutions are unable to fulfill the promise of a real tactile feel, and at the same time are complicated and have a significant risk of imparting incorrect information, which leads to misinterpretations and actions.

In other words, even though a 1:1 converted tactile feel of the tissue would be the general desideratum, the intention here is to initially enable force control for systems driven by servomotor that is equal to that of purely manually mechanically driven systems. The inventors have recognized that for motor-driven system movements, it is initially already of great assistance to the user if the user is able to control the effect of force as in the conventional, purely manually mechanically driven systems.

In systems where hand movements on the actuating unit are converted into system movements via servomotors, one aspect of the invention consists in particular of entirely dispensing with a force measurement of the counterforce of the tissue on the distal side of the endoscopic system.

In return, one or more force sensors are additionally integrated in the manually operable actuating unit in particular and measure the forces which the user exerts on the actuating unit for the purpose of controlling the system. By measuring the change in these forces exerted on the actuating unit by the user, the movement forces due to the motor-driven system movements are modified in comparable fashion.

Even though this design is comparatively simple, it surprisingly advantageously allows the user to vary the forces as in a conventional manual MIS system even in the case of motor-driven systems, and to adapt said forces by way of visual observation and control of the effect on the tissue, for example when gripping tissue. In particular, the user is able to meter a gripping force on the tissue by way of a manually applied operating force, in particular finger forces, and to monitor the effectiveness thereof visually, especially in real time. It is the insight of the inventors that it is not decisive whether the operating force exerted on the actuating unit or input unit actually precisely corresponds to the actuator force, in particular the jaw part gripping force. What is decisive is that the actuator force or gripping force can be changed by way of the operating force or closing force applied by the user.

This completely achieves the object.

In an advantageous configuration, the system is designed for a forceps function, the actuator comprises two jaw parts and the actuation of the actuator is an opening and closing of the jaw parts.

The assumption is made that the implementation of the invention in this technical context is particularly advantageous, especially in the case of grasping forceps. However, other types of system also come into question as a matter of principle, for example those which have a pair of scissors, a variable loop or a rotatable element as an actuator.

In a further advantageous configuration, the actuating unit comprises a force sensor designed to measure the operating force exerted on the actuating unit, at least in the end position.

The exerted operating force can be measured comparatively easily in this way.

In a further advantageous configuration, the force sensor comprises a bending element for measuring a deformation of the actuating unit.

This type of force measurement is robust and can be realized with comparatively little technical outlay. There is also the option of retrofitting existing systems with such a bending element, and hence of also realizing the invention in existing systems.

In a further advantageous configuration, the actuating unit comprises a position sensor designed to determine the current position of the actuating unit between the start position and the end position.

The current position of the actuating unit can be measured comparatively easily in this way.

In a further advantageous configuration, the limit value can be set by the user.

In particular, this provides the user with the option of implementing an adaptation with respect to the nature of the object with which the actuator is intended to interact. In this case, the user can choose a lower limit value in the case of a pressure-sensitive object than in the case of a less pressure-sensitive or harder object.

In a further advantageous configuration, the limit value is realized by restricting the current conducted to the actuator motor.

The limit value can be implemented comparatively easily in this way. This is particularly true if the specific practical application does not allow a direct measurement on the actuator, or if such a direct measurement is complicated.

In a further advantageous configuration, the limit value is realized by way of a slip coupling or an adjustable overload spring.

The limit value can be reliably implemented in this way by resorting to mechanical means. In this case, such an implementation can be realized in addition to other measures, for example a restriction of the current conducted to the actuator motor, in order to achieve redundancy and safety.

In a further advantageous configuration, the actuating unit comprises a first rigid grip member and a second movable grip member.

The object is also achieved by a method for driving an actuator of an endoscopic system, in particular an endoscopic system as described herein, an actuator motor, operatively connected to the actuator, a) being actuated depending on a current position of an actuating unit of the endoscopic system when the actuating unit is actuated between a start position and an end position and if an actuator force exerted by the actuator does not exceed a limit value, b) not being actuated any further when the actuating unit is actuated between the start position and the end position and if an actuator force exerted by the actuator reaches or exceeds the limit value, and c) being driven beyond the limit value, depending on an operating force exerted on the actuating unit, when the actuating unit is actuated in the end position.

It will be appreciated that the features mentioned above and the features yet to be explained below are applicable not only in the respectively specified combination but also in other combinations or on their own, without departing from the scope of the present invention. Further embodiments, and some of the advantages connected to these and further embodiments, are rendered clear and better understandable by the following detailed description which makes reference to the attached figures. Objects or parts thereof which are substantially the same or similar may be provided with the same reference signs. The figures are merely a schematic illustration of an embodiment of the invention. Further advantages arise from the following description of the drawing. An exemplary embodiment of the invention is depicted in the drawings. The drawings, the description, and the claims contain numerous features in combination. A person skilled in the art will advantageously also consider the features on an individual basis and combine them to form further advantageous combinations.

Exemplary embodiments of the invention are depicted in the drawing and are described in more detail in the following description. In the drawing:

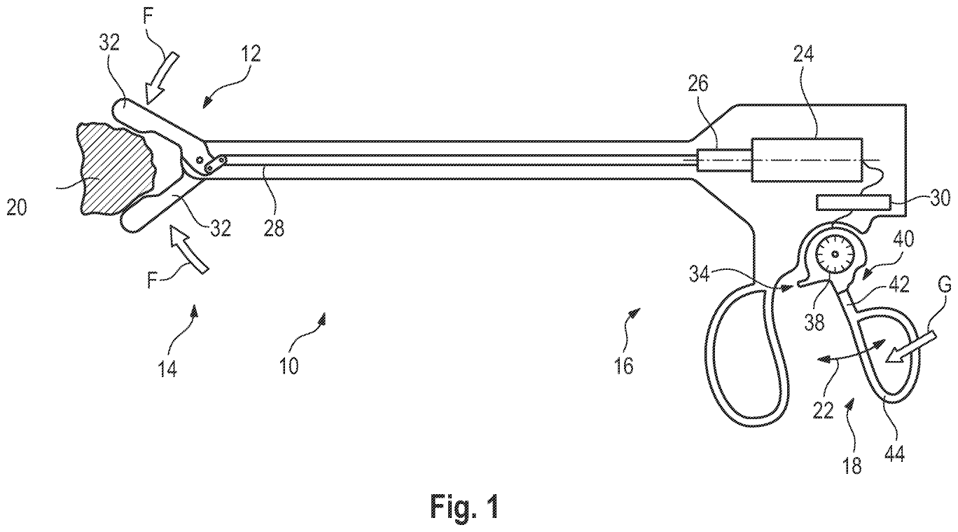

FIG. 1 shows an embodiment of an endoscopic system, and

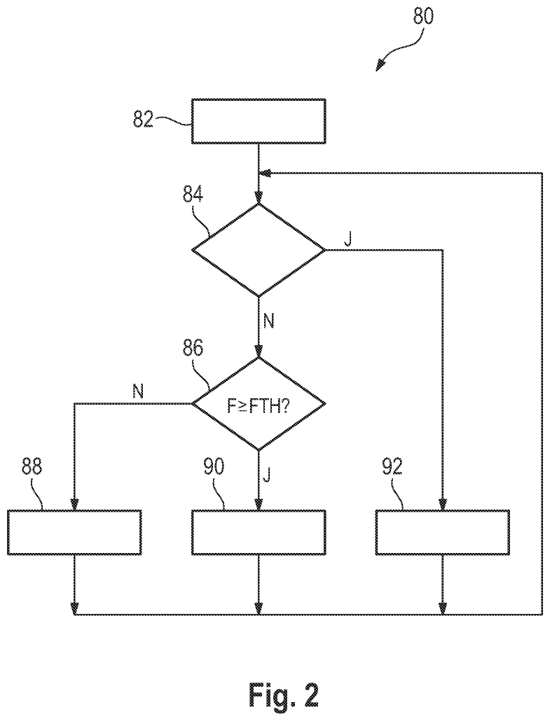

FIG. 2 shows an embodiment of a method for driving an actuator of an endoscopic system.

FIG. 1 shows an embodiment of an endoscopic system 10 comprising an actuator 12 at a distal end 14 of the system 10. In this case, the endoscopic system 10 is configured as an endoscopic instrument which, in the totality thereof, is guided manually by the surgeon. However, other configurations are also possible, for example as a robotic system, with the distal end 14 then being controlled by the surgeon but not being directly guided manually by the surgeon.

A manually operable actuating unit 18 for actuating the actuator 12 and choosing an actuator force F by means of which the actuator 12 is intended to act on an object 20 is arranged at a proximal end 16 of the system 10. The actuating unit 18 is displaceable between a start position and an end position, as indicated by a double-headed arrow 22, by virtue of the user exerting an operating force G on the actuating unit 18. On the basis of the principle of a force equalling a counterforce, the operating force G can also be felt by the user.

The endoscopic system 10 further comprises an actuator motor 24 which is designed to drive the actuator 12, in this case via a controllable slip coupling 26 and a tension/push rod 28. Further, a control unit 30 is present and designed to drive the actuator motor 24.

In a first operating state, in which the actuator force F exerted by the actuator 12 does not exceed a limit value FTH, the actuator 12 is actuated depending on a current position of the actuating unit 18 when the actuating unit 18 is actuated between the start position and the end position. This means that a continuous actuation of the actuating unit 18 leads to a continuous actuation of the actuator 12 for as long as the limit value FTH of the actuator force F exerted by the actuator 12 has not been reached.

Specifically, the actuating unit 18 is continuously actuated in the direction of the end position, the jaw parts 32 of the actuator 12 closing ever further as a result. In this operating state, the user feels only a small counterforce or no counterforce at all from the actuating unit 18.

In a second operating state, the actuator force F exerted by the actuator 12 has reached or exceeded the limit value FTH. As a rule, this occurs when the actuator 12 has come into contact with the object 20 and has started to act on the object 20. As a result, a continuous actuation of the actuating unit 18 no longer leads to a continuous actuation of the actuator 12. Specifically, the actuator 12 moves no further.

In the case of the jaw parts 32 shown here in exemplary fashion, this means that the jaw parts 32 are not closed any further but remain in the current position. This usually also leads to the actuator force F exerted by the jaw parts 32 remaining unchanged. In other words, there is no further actuation of the actuator 12 despite the user still continuously actuating the actuating unit 18 in the direction of the end position.

The actuating unit 18 has reached the end position in a third operating state. In particular, the end position can be realized by means of an end stop 34. Now, the actuator 12 is driven beyond the limit value FTH depending on an actuator force F actuated on the actuating unit 18. That is to say, the actuator force F exerted by the actuator 12 can now be increased in a finely metered fashion in the end position. In the embodiment shown here, the slip coupling 26 is closed further in order to enable the larger transfer of force desired at this juncture.

Specifically, the jaw parts 32 can now be closed even further, until the desired gripping situation is attained. In the process, the user can verify the desired success by way of an electronic visual display, which shows the operating region of the actuator 12. However, the user also receives direct haptic feedback at the same time because they are pressing the actuating unit 18 against the end stop 34 with the operating force G.

In the exemplary embodiment shown here, the displacement of the actuating unit 18 in the first and second operating states is sensed by means of a position sensor 38 and the operating force G at the actuating unit 18 is sensed in the third operating state by means of a force sensor 40 comprising a bending element 42. The actuating unit 18 has a grip member 44.

FIG. 2 shows an embodiment of a method 80 for driving an actuator 12 of an endoscopic system 10. The assumption is made here that the method 80 starts at step 82, in which the actuating unit 18 is in the start position and subsequently moved from the start position in the direction of the end position.

Whether the end position has been reached is checked in a step 84. Should this not be the case, the method 80 branches to step 86 via the N branch. Here, a check is carried out as to whether the actuator force F currently exerted by the actuator 12 has reached or exceeded the limit value FTH. Should this not be the case, the method 80 branches to step 88 via the N branch. This step 88 corresponds to the first operating state, in which a continuous actuation of the actuating unit 18 leads to a continuous actuation of the actuator 12.

Should the check in step 86 be positive, the method 80 branches to step 90 via the J branch. This step 90 corresponds to the second operating state, in which a continuous actuation of the actuating unit 18 no longer leads to a continuous actuation of the actuator 12.

Should the check in step 84 be positive, the method 80 branches to step 92 via the J branch. This step 90 corresponds to the third operating state, in which the actuator 12 is driven beyond the limit value FTH depending on an operating force G exerted on the actuating unit 18.

In one embodiment, the technical procedure is preferably as set forth below. First, the actuating unit 18 is moved from the start position in the direction of the end position. Since the grip member 44 can move freely, no, or only a minimal, operating force G can be felt on the actuating unit 18.

The actuator motor 24 is driven by way of the position sensor 38 and the control module 30, is moved in accordance with the transmission mechanism 26, 28, and closes the jaw parts 32, with the result that the object 20, in this case a tissue, is gripped.

On account of the small operating force G or closing force at the actuating unit 18, the actuator force F or the gripping force of the jaw parts 32 is significantly restricted by the control module 30 (e.g., by limiting the power of the motor force of the actuator motor 24 and/or by setting the slip coupling 26), with the result that the jaw parts 32 are only closed until the object 20 is gripped with a small actuator force F or gripping force. On the basis of the deformation of the object, which is to say a tissue deformation in particular, the user is able to identify the hardness or softness of the object 20 or tissue.

Since, on account of the small actuator force F or gripping force, the jaw parts 32 do not close any further when the object 20 is gripped, but the grip member 44 of the actuating unit 18 is freely movable, the user can fully close the grip member 44 up to the end stop 34. There is no change in the actuator force F or gripping force up until this point, since no, or only a minimal, operating force G or closing force acts on the force sensor 40 until then.

As soon as the actuating unit 18 is closed or has reached the end position, the user is able by way of the grip member 44 to apply an increased operating force G against the end stop 34. The latter is sensed by way of the force sensor 40 and the control module 30, by means of which the motor force of the actuator motor 24, and hence the actuator force F or gripping force on the object 20, is subsequently increased or readjusted in accordance with the operating force G at the actuating unit 18 manually applied by the user.

In preferred embodiments, different variants and embodiments are used for the force sensor system on the actuating unit 18 or input unit. In addition to different force sensors 40, this also includes a resilient yield when force is applied, which could facilitate the handling since a tactile position detection, as it were, assists with metering force during input, and a spring element in the grip member 44, with the result that the position sensor system in combination with a spring characteristic can also be used for the force measurement at the same time.

Other techniques for modifying the actuator force F or gripping force are used in preferred embodiments. Rather than modifying the actuator force F or a gripping force by way of a motor controller, in particular by means of load limiting, a change in the pretension of an overload spring can be controlled by way of a second motor, or the desired feedback can be provided by a sliding coupling, a transmission and/or a braking mechanism.

In preferred embodiments, an additional, more particularly distal position measurement is used to optimize the control. Thus, the actuating unit 18 or input unit can be blocked as soon as the actuator 12, in particular the jaw parts 32, no longer move(s). This can preferably be achieved by way of a latching mechanism, by way of a motor force or motor counterforce, and/or a braking mechanism.

Moreover, it may be preferable to integrate a spring element in the drivetrain. This enables a measurement of the spring travel, and the actuator force F or gripping force can then be set by way of the motor movement in combination with a spring characteristic. It is preferably also possible to employ different measurement methods, in particular an optical position measurement by way of an imaging system, the images of which are evaluated in respect of a system recognition and/or movement detection, preferably with the aid of artificial intelligence.

An exemplary embodiment of the invention is depicted in the drawings. The drawings, the description, and the claims contain numerous features in combination. A person skilled in the art will advantageously also consider the features on an individual basis and combine them to form further advantageous combinations. An endoscopic system 10 comprises an actuator 12 at a distal end 14 of the system 10, a manually operable actuating unit 18 at a proximal end 16 of the system 10, for actuating the actuator 12 and choosing an actuator force F by means of which the actuator 12 is intended to act on an object 20, the actuating unit 18 being displaceable between a start position and an end position, an actuator motor 24 designed to drive the actuator 12, and a control unit 30 designed to drive the actuator motor 24 initially on the basis of a position sensor 38 without exceeding a limit value FTH and then on the basis of a force sensor 40. Further, a method for driving an actuator 12 of an endoscopic system 10 is disclosed.

1. An endoscopic system, comprising:
an actuator at a distal end of the system;
a manually operable actuating unit at a proximal end of the system, configured to actuate the actuator and select an actuator force configured to allow the actuator to act on an object, the actuating unit being displaceable between a start position and an end position,
an actuator motor configured to drive the actuator to reach a desired position, and
a control unit configured to:
a) actuate the actuator motor depending on a current position of the actuating unit when the actuating unit is actuated between the start position and the end position and if an actuator force exerted by the actuator does not exceed a limit value (FTH),
b) not actuate the actuator motor any further when the actuating unit is actuated between the start position and the end position and if an actuator force exerted by the actuator reaches or exceeds the limit value (FTH), and
c) drive the actuator motor beyond the limit value (FTH), depending on an operating force exerted on the actuating unit, when the actuating unit is actuated in the end position.

2. The endoscopic system as claimed in claim 1, wherein the system is configured for a forceps function, the actuator comprises two jaw parts and the actuation of the actuator an opening and closing of the jaw parts.

3. The endoscopic system as claimed in claim 1, wherein the actuating unit comprises a force sensor configured to measure the operating force exerted on the actuating unit, at least in the end position.

4. The endoscopic system as claimed in claim 3, wherein the force sensor comprises a bending element configured to measure a deformation of the actuating unit.

5. The endoscopic system as claimed in claim 1, wherein the actuating unit comprises a position sensor configured to determine the current position of the actuating unit between the start position and the end position.

6. The endoscopic system as claimed in claim 1, wherein the limit value (FTH) can be set by the user.

7. The endoscopic system claimed in claim 1, wherein the limit value (FTH) is realized by restricting the current conducted to the actuator motor.

8. The endoscopic system claimed in claim 1, wherein the limit value (FTH) is realized by way of a slip coupling or an adjustable overload spring.

9. The endoscopic system as claimed in claim 1, wherein the actuating unit comprises a movable grip member.

10. A method to drive an actuator of an endoscopic system, an actuator motor, operatively connected to the actuator,
a) being actuated depending on a current position of an actuating unit the endoscopic system when the actuating unit is actuated between a start position and an end position and when an actuator force exerted by the actuator does not exceed a limit value,
b) not being actuated any further when the actuating unit is actuated between the start position and the end position and if an actuator force exerted by the actuator reaches or exceeds the limit value, and
c) being driven beyond the limit value (FTH), depending on an operating force exerted on the actuating unit, when the actuating unit is actuated in the end position.
